(19) United States
(12) Patent Application Publication
(10) Pub. No.: US 2004/0138707 A1
(43) Pub. Date: Jul. 15, 2004

(54) Anchor removable from a substrate

(76) Inventor: E. Skott Greenhalgh, Wyndmoor, PA (US)

Correspondence Address: John A. Chionchio, Esquire, Synnestvedt & Lechner LLP, Suite 2600, 1101 Market Street, Philadelphia, PA, 19107-2950, US

(21) Appl. No.: 10/342,008

(22) Filed: Jan. 14, 2003

(51) Int. Cl. A61B 17/04

(52) U.S. Cl. 606/232

(57) ABSTRACT

An anchor removably positionable within a cavity in a substrate is disclosed. The anchor has an anchor body from which a plurality of resiliently biased legs extend. The legs are movable between a first position within the envelope of the anchor body diameter, and a second position extending radially beyond the anchor body diameter. The legs are biased to assume the second position when unconstrained. Free ends of the legs have engagement surfaces receivable within an insertion tool for constraining the legs into the first position. An attachment extends from the anchor body for connection to an item to be attached to the substrate. Insertion of the anchor into a distal portion of the cavity having a reduced diameter forces the legs into the first position, allowing engagement of the insertion tool with the legs and removal of the anchor from the cavity.

### FIELD OF THE INVENTION

The invention concerns an anchor positionable within a substrate for attaching an item to the substrate and a method of positioning the anchor within the substrate and removing the anchor from the substrate.

### BACKGROUND OF THE INVENTION

It is often advantageous to use an anchor to effect a strong and reliable connection to a substrate. This is especially true when materials of vastly different stiffness are to be joined together. For example, in the human body, tendons comprise fibrous cords of soft tissue which connect muscle tissue to bone tissue. Injuries to the body involving a torn tendon require that the soft, fibrous tissue comprising the tendon be reattached to the hard, substantially rigid tissue forming the bone. To repair such an injury, an anchor is fixed to the bone near the position where the tendon is to be attached and a suture is attached to the anchor. The suture is sewn into the tendon to hold it in place against the bone so that it may heal and reattach itself to the bone. The anchor provides a device incorporating different forms of attachment appropriate to the characteristics of the particular materials being joined. The anchor also provides a device which spreads what would otherwise be a concentrated load over a larger area of the substrate, thereby reducing the stress in the substrate and enabling, for example, a suture to transmit large loads from a tendon to the bone thereby keeping the tendon in position to promote healing.

While anchors are generally known and used to repair severed tendons or to fix plates assisting the healing of broken bones, they are not easily removable or repositionable once implanted. Removable anchors would provide an advantage over fixed anchors as they would allow for the correction of mistakes or otherwise unforeseen difficulties in the placement of the anchor which, if not corrected, may adversely affect the rate of healing and the effectiveness of the repair.

### SUMMARY AND OBJECTS OF THE INVENTION

The invention concerns an anchor positionable within a cavity in a substrate for attaching an item to the substrate. The cavity is accessible through an aperture in a surface of the substrate. The aperture has a smaller diameter than the cavity. The anchor is removably attachable to an insertion tool for positioning the anchor within the cavity.

The anchor comprises an anchor body having a diameter substantially equal to the aperture and sized to pass through it. An attachment is fixed to the anchor body for attaching the item to the anchor. The attachment projects away from the anchor body so that it can extend outwardly through the aperture. The attachment may, for example, be a suture or a finger to which a bone plate may be bolted. A plurality of resilient, flexible legs are attached to the anchor body in spaced relation circumferentially therearound. Each of the legs has a free end movable between a first position located within the anchor body diameter and a second position extending radially outwardly beyond the anchor body diameter. The legs are resiliently biased into the second position. An engagement surface is positioned on each of the legs at their free ends. Each of the engagement surfaces faces radially outwardly and is located within the anchor body diameter when the legs are in the first position. In this position, the engagement surfaces are engageable with the insertion tool. The legs are movable outwardly under the resilient biasing into the second position upon disengagement of the insertion tool with the engagement surfaces.

In one preferred embodiment, the engagement surfaces are defined by respective notches positioned in the free ends of the legs. In another embodiment, the legs are tapered at the free ends to define the engagement surfaces.

The anchor as described above is positionable within a cavity in bone matter. This is useful for attaching an item, such as a tendon, ligament or a bone plate to the bone matter. The bone matter comprises a relatively hard cortical layer overlying a relatively soft cancellous layer. The legs of the anchor are sufficiently flexible so as to be movable radially inwardly upon engagement of the legs with the cancellous layer during insertion of the anchor body into the distal cavity portion, the anchor thereby having substantially the same diameter as the distal cavity portion when sufficiently inserted into the distal cavity portion. This flexibility will allow the anchor to be easily removable from the bone matter as described below.

The invention also includes an anchor kit for removably positioning the anchor within a cavity in a substrate. The cavity is accessible through an aperture in a surface of the substrate and has a distal portion positioned distally to the surface, a middle portion positioned between the distal portion and the surface, and a proximal portion positioned proximally to the surface. The middle cavity portion has a diameter greater than the distal and proximal cavity portions, and an inwardly extending shoulder is thus formed between the middle and proximal cavity portions.

The anchor kit comprises an anchor body having a plurality of lengthwise extending legs attached to it. As described above, each of the legs has a free end resiliently biased to move outwardly and engage the shoulder upon insertion of the anchor body into the middle cavity portion. To facilitate removal of the anchor from the cavity, the legs are inwardly movable upon insertion of the anchor body into the distal cavity portion. The anchor also has a contact surface positioned between the legs of the anchor body. The contact surface faces outwardly from the cavity when the anchor is positioned therein. The kit includes an insertion tool, preferably comprising an elongated tube sized to pass through the aperture and substantially fill the proximal and distal cavity portions. The tube has a longitudinal bore therethrough sized to receive the free ends of the legs when the anchor body is positioned within distal cavity portion. The kit also includes an elongated rod sized to pass through the aperture, the rod having an end engageable with the contact surface. The rod is used to force the anchor body into the distal cavity portion. This causes the substrate surrounding the distal cavity portion to engage and thereby moving the legs inwardly. The tube, being positionable within the cavity with the bore aligned with the anchor body, can engage the free ends of the legs which are movable into the bore. The legs are thus constrained inwardly by the tube into the second position, allowing the tube to be removed from the cavity with the anchor body therein.

The invention also contemplates a method of inserting and removing an anchor into and from a cavity in a substrate. The insertion method comprises the steps of:

(A) engaging the free ends of the legs with the end of the insertion tool thereby holding the legs inwardly;

(B) inserting the anchor and the insertion tool through the aperture and into the cavity;

(C) disengaging the insertion tool end from the legs, the legs moving outwardly under the resilient biasing; and

(D) withdrawing the insertion tool from the cavity.

The removal method comprises the steps of:

(A) pushing the anchor body into the distal cavity portion sufficiently to move the legs inwardly;

(B) inserting an elongated tool into the cavity, the tool having an end for receiving the legs;

(C) engaging the tool end with the legs, the tool receiving and constraining the legs from moving outwardly; and

(D) withdrawing the tool from the cavity with the legs of the anchor body engaged with the tool.

It is an object of the invention to provide an anchor which can be easily inserted and removed from a cavity in a substrate.

It is another object of the invention to provide an anchor which provides a high pull-out strength by reducing stress in the substrate.

It is again another object of the invention to provide an anchor effective at joining dissimilar materials together.

It is yet another object of the invention to provide an anchor suitable for use in bone matter.

It is still another object of the invention to provide a kit for inserting and removing anchors into and from a substrate.

It is furthermore another object of the invention to provide a method for inserting and removing anchors into and from a substrate.

These and other objects and advantages of the invention will become apparent upon consideration of the drawings and the detailed description of preferred embodiments.

### BRIEF DESCRIPTION OF THE DRAWINGS

FIG. 1 is a perspective view of an anchor according to the invention;

FIGS. 2 through 8 and 8A illustrate insertion and removal of the bone anchor shown in FIG. 1 into and from a substrate, FIG. 8A depicting a cross sectional view taken along lines 8A-8A from FIG. 8;

FIG. 9 is a sectional view of anchors according to the invention used to repair a fracture in bone matter;

FIG. 10 is a cross-sectional view taken along lines 10-10 of FIG. 9;

FIGS. 11 through 18 are perspective views of alternate embodiments of anchors; and

FIG. 19 is a sectional view of an anchor embodiment being inserted into a cavity.

### DETAILED DESCRIPTION OF THE PREFERRED EMBODIMENTS

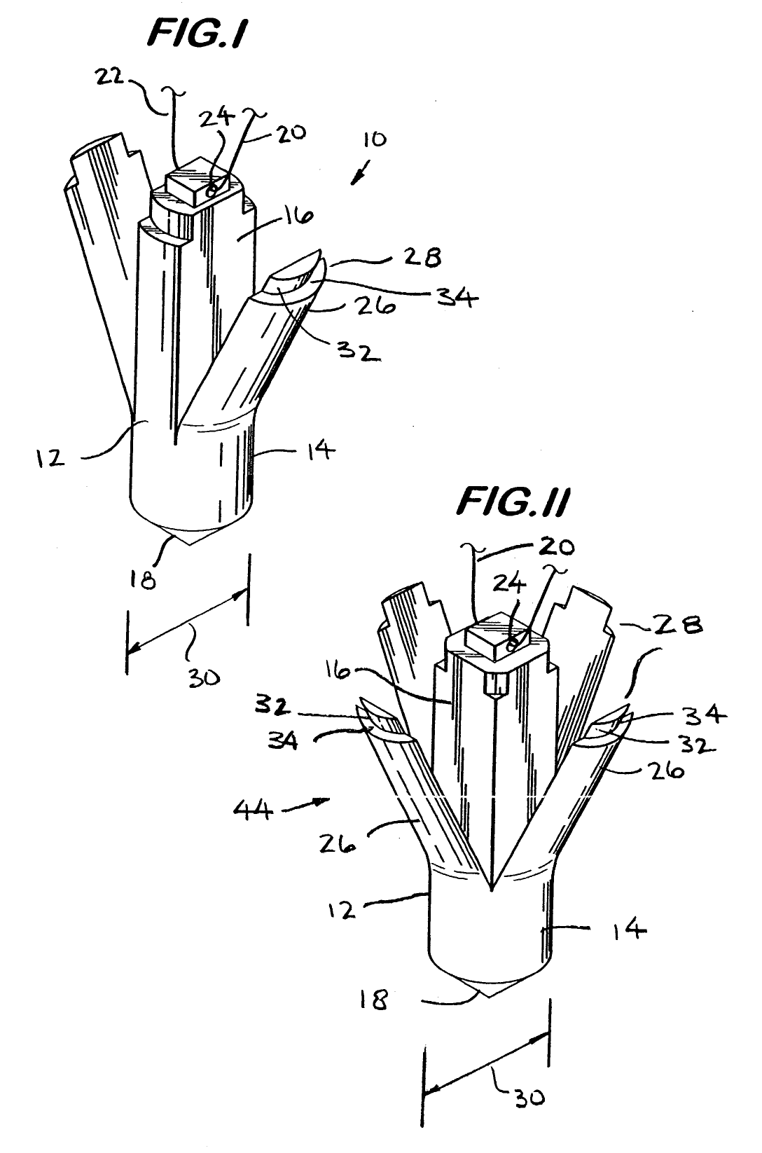

FIG. 1 shows an anchor 10 removable from a substrate according to the invention. Anchor 10 comprises an anchor body 12 formed of a head 14 and a tail 16 extending lengthwise away from the head 14. Head 14 may have a tapered end 18 to facilitate insertion of the anchor through an aperture and into a cavity. An attachment 20 is fixed to the tail 16 and projects from the anchor body. In the particular embodiment illustrated in FIG. 1, the attachment comprises a filamentary member 22, for example, a suture. Filamentary attachments such as 22 are preferably attached to tail 16 using an eye 24 mounted on the tail. Other forms of attachment are feasible, and examples are described below.

A plurality of resilient, flexible legs 26 are attached to the anchor body 12 in spaced relation circumferentially there around. Each leg 26 has a free end 28 movable between a first position located within the diameter 30 of the anchor body 12 (see FIGS. 4 and 5) and a second position extending radially outwardly beyond the anchor body diameter 30 as shown in FIG. 1. Legs 26 are preferably biased to move into the second position extending outwardly from the anchor body in the absence of restraining forces holding them within the anchor body diameter. To achieve the desired resilience and biasing, it is advantageous to form the anchor 10 from resilient, flexible material having a relatively high yield stress. Nitinol is preferred, but elgiloy, stainless steel, titanium and polymer materials such as polyester, polystyrene, polypropylene, PEEK, polyethylene and polytetrafluoroethylene are also feasible. When used as a surgical implant, for example, as a bone anchor, the material comprising the anchor 10 should also be compatible with living tissue. Biodegradable materials such as polyglycolic acid, polylactic acid, PLLA, PDO and PCL may also be used to form the anchor.

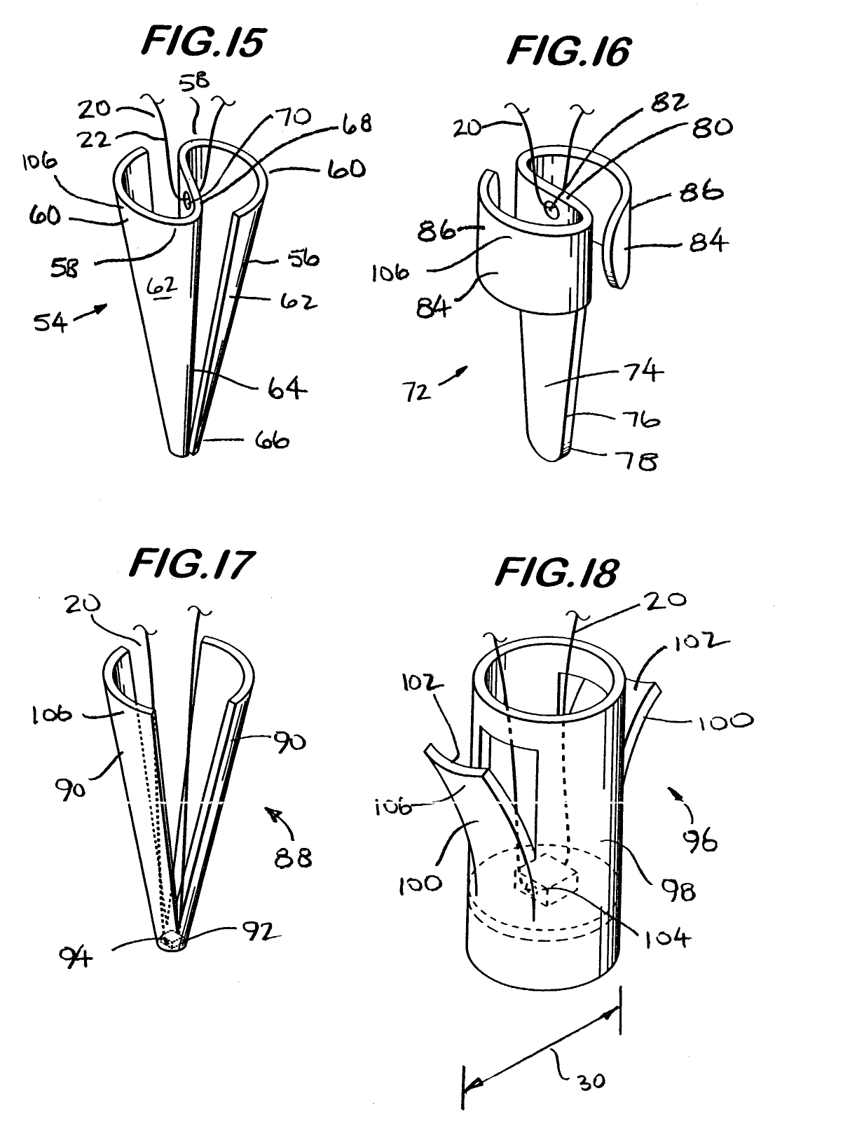

Each leg 26 has an engagement surface 32 positioned at its free end 28. Engagement surfaces 32 face outwardly and are positioned within the anchor body diameter 30 when the legs 26 are in the first position. The engagement surfaces 32 engage an insertion tool, described below, for inserting and removing the anchor 10 in a cavity. In the embodiment illustrated in FIG. 1, the engagement surfaces 32 are each defined by respective notches 34 formed in each of the free ends 28 of the legs 26. Other forms of engagement surfaces include outer surfaces 36 of tapered legs 38 shown in FIG. 19 as well as the outer surfaces 40 of multiple legs 42 as shown in FIG. 13.

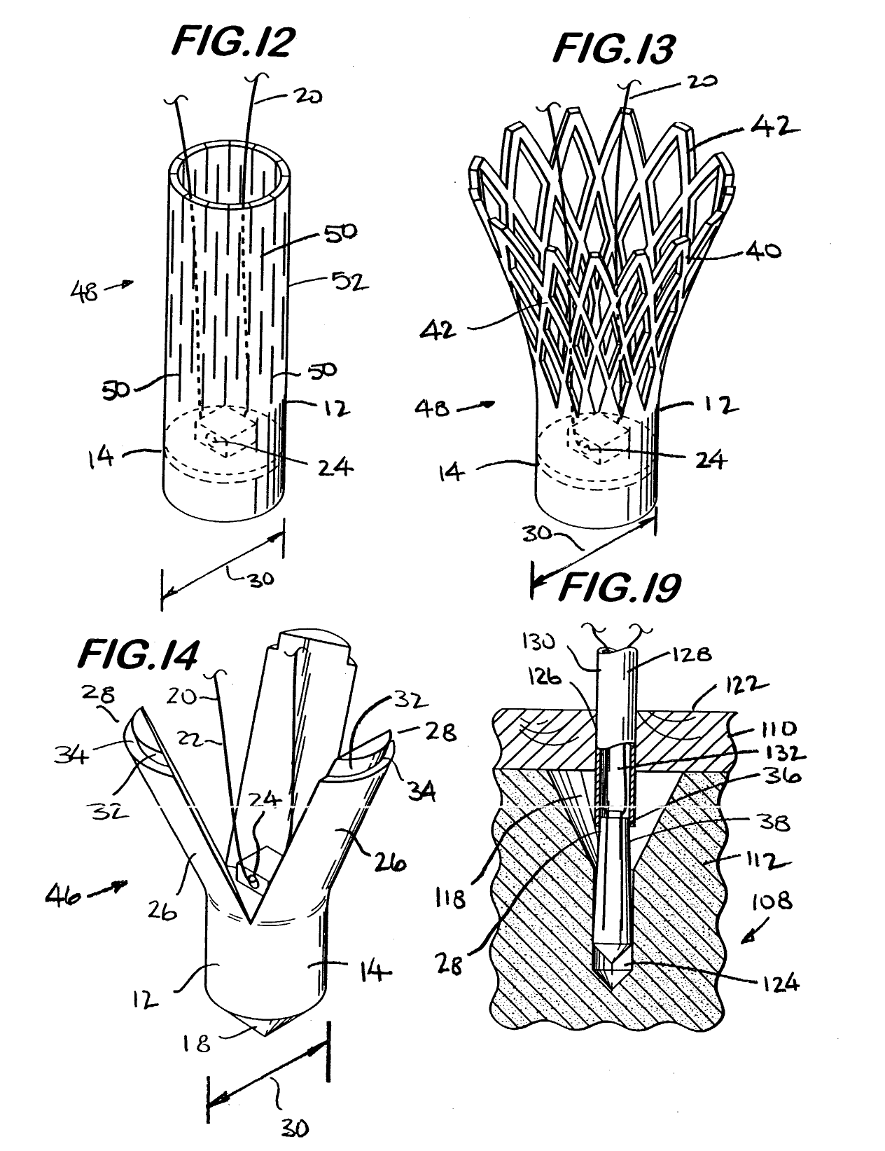

FIG. 11 illustrates an anchor embodiment 44 having four legs 26, each with notches 34 forming an outwardly facing engagement surface 32. Yet another anchor embodiment 46 is illustrated in FIG. 14, wherein three legs 26, each having a notch 34 defining an engagement surface 32, extend from a head 14 having a tapered end 18. Attachment 20, in the form of a filamentary member 22, is attached directly to head 14 via eye 24. FIGS. 12 and 13 show an anchor embodiment 48 having multiple interconnected legs 42 formed by cutting a plurality of slits 50 in a tubular blank 52 and then splaying the legs 42 outwardly by compressing and expanding them beyond the diameter 30 of the anchor 48. Attachment 20 is fixed to the head 14 by an eye 24 located within the tubular blank 52. Slits 50 may be cut by a saw or a laser, and the legs 42 are resiliently biased into the outwardly splayed position by a means appropriate to the material comprising the tubular blank. For example, metals may be cold-worked over an appropriately shaped mandrel, and polymeric materials may be biased into position by heat or chemical means.

FIGS. 15-18 show embodiments of anchors according to the invention formed from flexible, resilient sheet material. FIG. 15 shows an anchor embodiment 54 wherein the sheet 56 has reverse curves 58 disposing the sheet into oppositely curved segments 60 forming legs 62 extending from the anchor body 64. Oppositely curved segments 60 give the anchor 54 an “S” shaped cross section. The head 66 is tapered to facilitate entry into a cavity, and the tail 68 has an integral eye 70 for receiving attachment 20 in the form of a filamentary member 22.

FIG. 16 shows an anchor embodiment 72 wherein the anchor body 74 is formed from a flat sheet portion 76, the head 78 being tapered and the tail 80 having an integral eye 82 for receiving an attachment 20. Legs 84 are formed by reverse curved sheet portions 86 positioned near the tail 80.

FIG. 17 shows an anchor embodiment 88 having legs 90 arcuately curved with concave portions facing inwardly of the anchor body 92. An eye 94 is positioned at the end of the anchor body 92 to receive an attachment 20. The anchors illustrated in FIGS. 15-17 are shown with the legs in the second position. For these anchor embodiments the phrase “outside of the anchor body diameter” describing the leg position means the legs are expanded outwardly to a diameter greater than the legs assume in the first position, the first position of the legs defining the anchor body diameter for these embodiments.

FIG. 18 shows another anchor embodiment 96 having a tubular anchor body 98 with legs 100 arcuately curved with concave portions 102 facing inwardly of the anchor body 98. An eye 104 is positioned within the anchor body 98 for receiving attachment 20. In each of the anchor embodiments shown in FIGS. 15-17 engagement surfaces comprise the outer surfaces 106 of the legs.

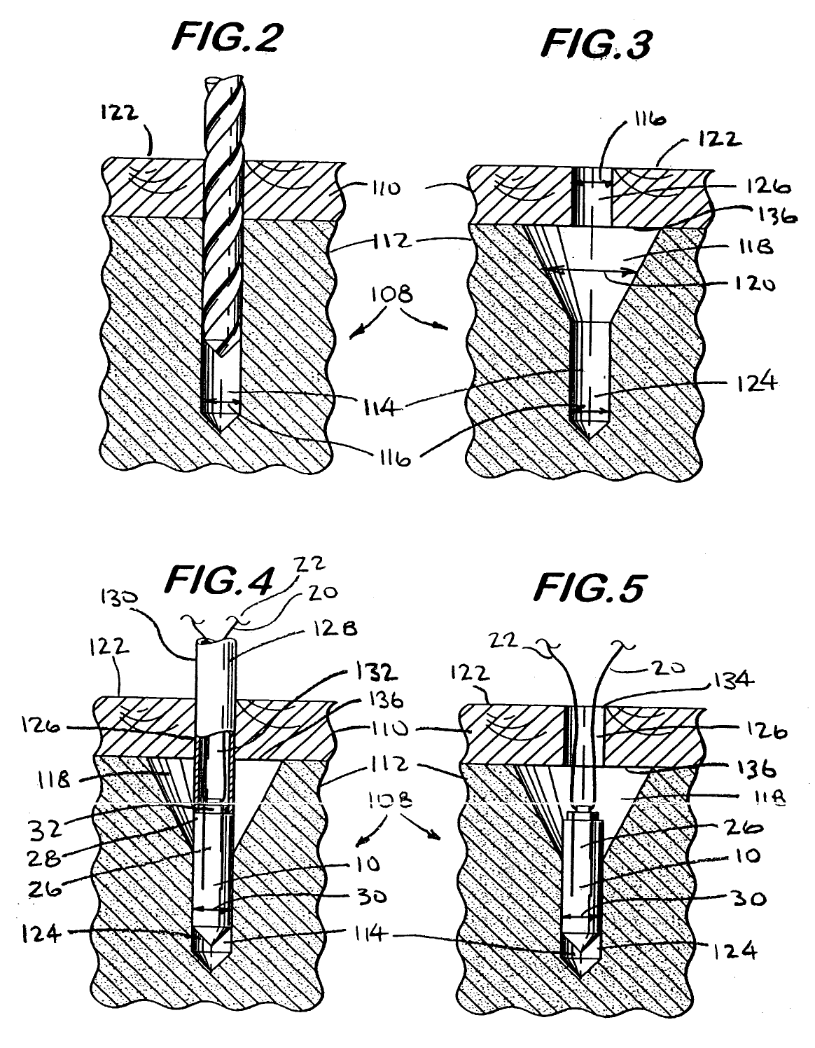

FIGS. 2-8 illustrate how an anchor according to the invention is inserted into and removed from a substrate 108. By way of example, substrate 108 may be bone matter comprising a hard cortical tissue layer 110 overlying a softer cancellous tissue layer 112. As shown in FIG. 2, a cavity 114 having a diameter 116 is formed in the substrate, preferably by drilling through the cortical and cancellous layers 110 and 112. Next a middle portion 118 of the cavity 114 is enlarged to a greater diameter 120 as shown in FIG. 3. The shape of the enlarged middle portion 118 is shown to be conical, but it could be any practical shape such as cylindrical, or even a reversed conical shape to that shown. Preferably, the middle cavity portion 118 is in spaced relation to the surface 122 of the substrate 108. The cavity 114 thus comprises a distal portion 124 having a diameter 116, a middle (enlarged) portion 118 having a larger diameter 120, and a proximal portion 126 having a diameter 116 substantially equal to the diameter of the distal portion 124.

With the cavity 114 prepared, anchor 10 is then inserted as shown in FIG. 4. Insertion is effected by means of an insertion/removal tool 128, preferably in the form of a tube 130 having a bore 132 sized to receive the engagement surfaces 32 on the free ends 28 of legs 26. (FIG. 19 shows an alternate embodiment with engagement surfaces formed by the outer surfaces 36 of tapered legs 38, the taper allowing the free ends 28 to be received within the tube bore 132 for insertion and removal as described herein.) Again with reference to FIG. 4, legs 26 are resiliently deflected to the first position for initial engagement with the bore 132, the tube 130 providing the restraint holding the legs against the biasing forces normally expanding the legs into the outwardly deflected second position. If necessary, tension may be applied to attachment 20 to hold the anchor 10 in engagement with the tube 130. Preferably, the diameter 30 of anchor 10 when legs 26 are held in the first position shown in FIG. 4 is approximately equal to the diameter 116 of the proximal and distal cavity portions but sized to pass through those portions for insertion into cavity 114. As shown in FIG. 5, the anchor 10, when inserted into the distal cavity portion 124, remains in the first position even after it is released from the tube 130. Release of anchor 10 from tube 130 may be effected, for example, simply by inserting a rod (not shown) through the bore 132 and pushing the anchor out of engagement with the bore. The stiffness of the legs 26 is such that the stiffness of the substrate layer, in this case the cancellous layer 112, in which the anchor is positioned, is sufficient to constrain the legs from taking the second, outwardly deflected position.

Attachment 20, in the form of filamentary member 22 extends from the anchor 10 through an aperture 134 in the surface 122 of the substrate 108. When the anchor 10 is positioned in the middle portion 118 of cavity 114 as shown in FIG. 6, either by drawing it out of the distal portion 124 by tensioning attachment 20 or by releasing it from the tube 130 while it is in the middle portion, legs 26, now unrestrained, splay outwardly into the second position and engage a shoulder 136 positioned between the substrate surface 122 and the middle cavity portion 118. The anchor 10 may now be used to attach items to the substrate. For example, the filamentary member 22 may be used to suture a tendon to the bone matter. The biasing forces within the legs 26 ensure that the legs deflect outwardly to engage the substrate shoulder 136 over a relatively large area of the cortical layer 110 thereby distributing the load and reducing the levels of stress in this layer resulting from tension being applied to the attachment 20. Thus, the anchor is able to resist relatively high pull-out forces before failing.

The shape of the cavity 114 combined with the stiffness of the legs 26 allows the anchor 10 to be removed from the cavity. Removal is illustrated in FIGS. 7, 8 and 8a wherein a push-piece in the form of an elongated rod 138 engages a contact surface 140, preferably positioned on the tail 16 of the anchor body 12. If a tail is not present as in some anchor embodiments, the contact surface is positioned directly on the head or other convenient part of the anchor body. Contact surface 140 faces outwardly from the cavity 114 and receives the rod 138. Pressure is applied forcing the anchor 10 into the distal cavity portion 124 as indicated by arrow 142. Because the diameter 116 of the distal cavity portion 124 is approximately equal to the diameter 30 of the anchor 10 with the legs 26 in the first position, and the legs are sufficiently flexible to be forced together by contact with the substrate 108 in the distal cavity portion 124, the legs 26 move inwardly into the second position as the anchor 10 is forced into the distal cavity portion 124. With legs 26 in the second position the engagement surfaces 32 on the free ends 28 are configured to be engaged by the tube bore 132. The tube 130 is then inserted into the cavity 114 so that the bore can capture engagement surfaces 32 as shown in FIG. 8A. Tension is placed on attachment 20 to hold the anchor in engagement with the tube 130, and, as shown in FIG. 8, the tube along with the anchor 10 may be withdrawn in the direction indicated by arrow 144 past shoulder 136 and out of the cavity 114.

It may be convenient to supply anchors, insertion/removal tools and push rods as a kit to ensure proper compatibility between the various components.

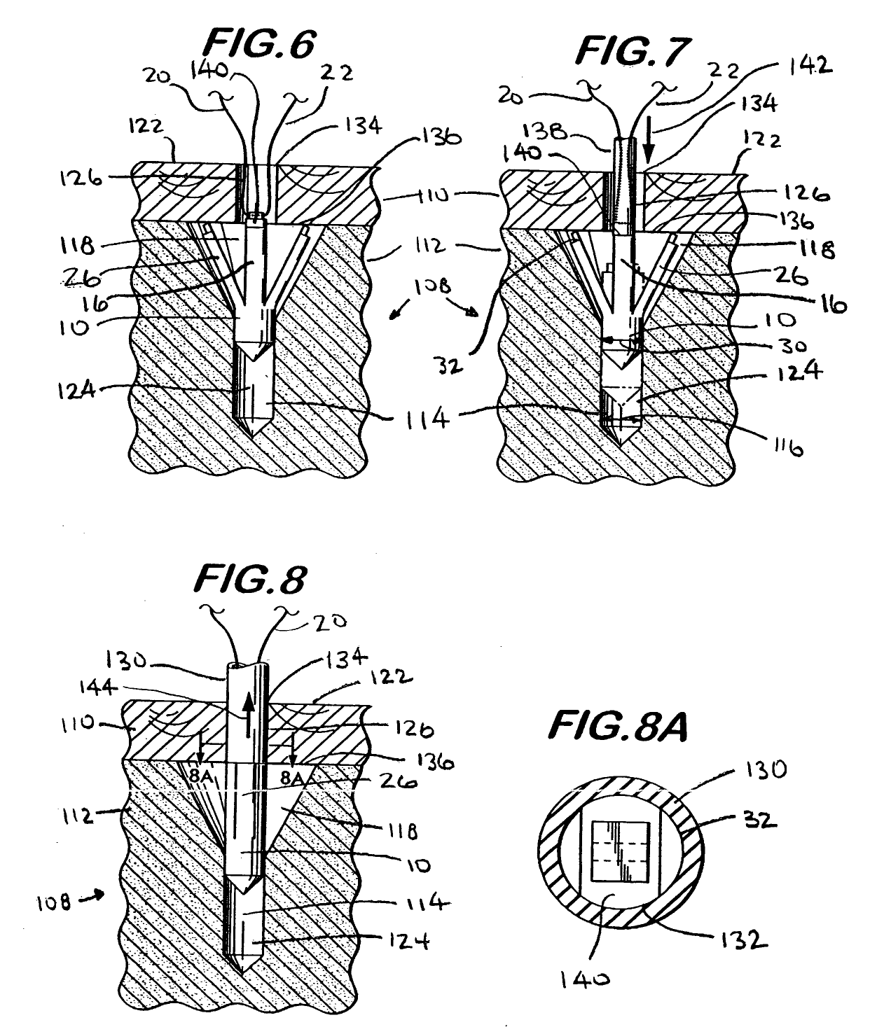

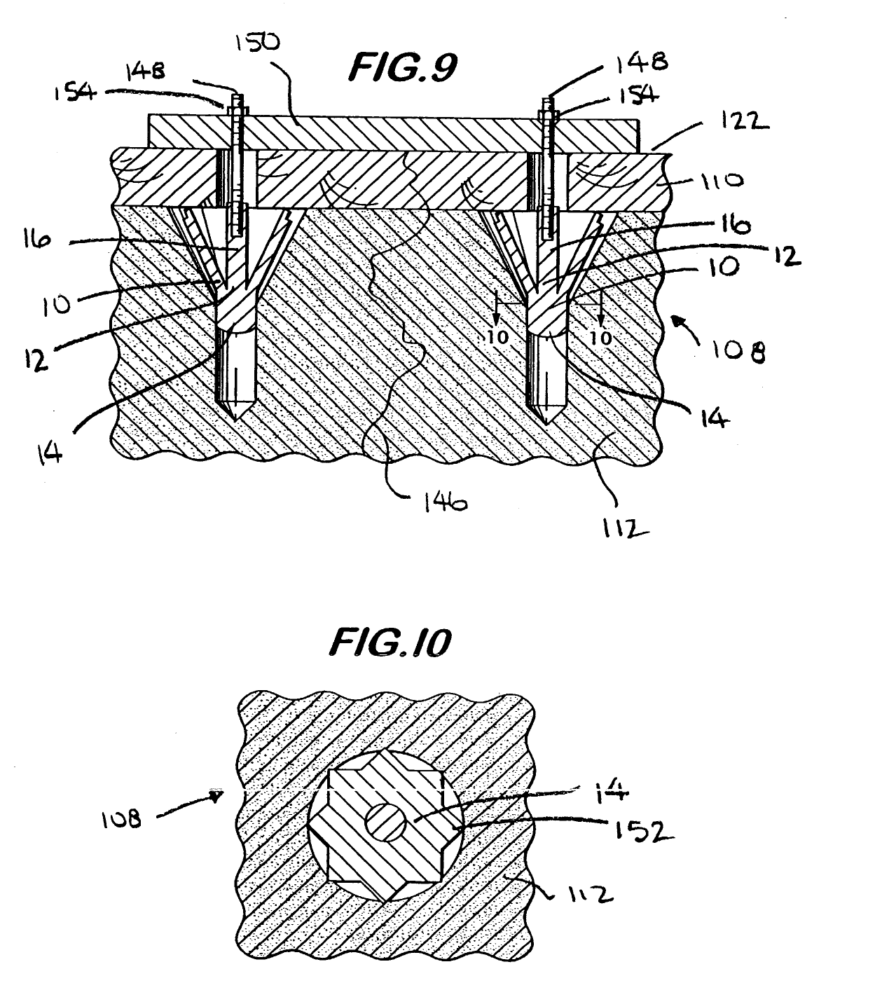

FIG. 9 shows anchors 10 used to repair a bone fracture 146 in the bone matter substrate 108. In this embodiment, the attachments comprise threaded studs 148 which extend like fingers from the tails 16 of the anchor bodies 12. Studs 148 project above surface 122 and engage an item, such as a bone plate 150 used to hold the bone matter together at the fracture 146 to promote proper healing. As the studs are threaded, it may be necessary to position radially extending serrations 152 on the anchor heads 14 as illustrated in FIG. 10. The serrations 152 engage the bone matter within the cavity 114 and prevent relative rotation of the anchors when nuts 154 are applied to the studs 148 to secure the bone plate 150 in place.

Anchors according to the invention provide a device for attaching items to a substrate and provide a strong and dependable attachment while still preserving the ability to conveniently remove and reposition the anchors as necessary without damage to hardware or trauma to living tissue, such as bone matter.

What is claimed is:

1. An anchor positionable within a cavity in a substrate for attaching an item to said substrate, said cavity being accessible through an aperture in a surface of said substrate, said aperture having a smaller diameter than said cavity, said anchor being removably attachable to an insertion tool for positioning said anchor within said cavity, said anchor comprising:
an anchor body having a diameter sized to pass through said aperture;
an attachment fixed to said anchor body for attaching said item thereto, said attachment projecting away from said anchor body so that said attachment can extend outwardly through said aperture;
a plurality of resilient, flexible legs attached to said anchor body in spaced relation circumferentially therearound, each of said legs having a free end movable between a first position located within said anchor body diameter and a second position extending radially outwardly beyond said anchor body diameter, said legs being resiliently biased into said second position; and
an engagement surface positioned on each of said legs at said free ends thereof, each of said engagement surfaces facing radially outwardly and being located within said anchor body diameter when said legs are in said first position and thereby engageable with said insertion tool, said legs movable outwardly under said resilient biasing into said second position upon disengagement of said insertion tool with said engagement surfaces.

2. An anchor according to claim 1, wherein said engagement surfaces are defined by respective notches positioned in said free ends of said legs.

3. An anchor according to claim 1, wherein said legs are tapered at said free ends thereby defining said engagement surfaces.

4. An anchor according to claim 1, wherein said attachment comprises an elongate finger extending lengthwise from said anchor body between said legs, said finger being sized so as to project above said surface for engagement with said item when said anchor body is positioned within said cavity.

5. An anchor according to claim 1, wherein said attachment comprises a filamentary member attached to said anchor body and extending lengthwise therefrom.

6. An anchor according to claim 1, wherein said anchor body comprises a head portion forming one end of said anchor body, and a tail portion extending lengthwise from said head portion and positioned between said legs, said legs extending from said head portion lengthwise along said tail portion.

7. An anchor according to claim 6, wherein said tail portion is adapted to extend outwardly through said aperture thereby comprising said attachment.

8. An anchor according to claim 6, wherein said head portion is tapered so as to facilitate insertion of said anchor body into said cavity.

9. An anchor according to claim 6, wherein said head portion has radially extending serrations preventing rotation of said anchor body within said cavity.

10. An anchor according to claim 6, wherein said tail portion has an eye therein for receiving a suture as said attachment.

11. An anchor according to claim 6, wherein said legs are adapted to be received within an end of a tube comprising said insertion tool, said tube being sized to hold said legs radially inwardly against said biasing thereby allowing said anchor to pass through said aperture and into said proximal and distal cavity portions, said legs movable outwardly under said resilient biasing upon disengagement of said tube end from said legs.

12. An anchor according to claim 11, wherein each of said legs has an outwardly facing notch positioned at said free end thereof, said notches adapting said legs for being received within said tube.

13. An anchor according to claim 11, wherein each of said legs is tapered at said free end thereof, said tapering of said legs adapting said legs for being received within said tube.

14. An anchor according to claim 1, wherein said anchor body comprises a head portion forming one end of said anchor body, said legs extending from said head portion with said free ends positioned distally to said head portion, and an eye formed in said head portion for receiving a suture, said suture comprising said attachment.

15. An anchor according to claim 14, wherein said head portion is tapered so as to facilitate insertion of said anchor body into said cavity.

16. An anchor according to claim 14, wherein said legs are adapted to be received within an end of a tube comprising said insertion tool, said tube being sized to hold said legs radially inwardly against said biasing thereby allowing said anchor body to pass through said aperture and into said proximal and distal cavity portions, said legs movable radially outwardly upon disengagement of said tube end from said legs.

17. An anchor according to claim 16, wherein each of said legs has an outwardly facing notch positioned at said free end thereof, said notches adapting said legs for being received within said tube.

18. An anchor according to claim 16, wherein each of said legs is tapered at said free end thereof, said tapering of said legs adapting said legs for being received within said tube.

19. An anchor according to claim 1, wherein each of said legs comprise a curved sheet of flexible material.

20. An anchor according to claim 19, wherein said legs are arcuately curved with concave portions facing inwardly of said anchor body.

21. An anchor according to claim 19, wherein said anchor body comprises a sheet of flexible material having an S-shaped cross section with oppositely disposed curved segments forming said legs.

22. An anchor positionable within a cavity in bone matter for attaching an item to said bone matter, said bone matter comprising a relatively hard cortical layer overlying a relatively soft cancellous layer, said cavity being accessible through an aperture in a surface formed by said cortical layer and having a distal portion positioned in said cancellous layer distally to said surface, a middle portion positioned between said distal cavity portion and said surface, and a proximal portion positioned within said cortical layer proximally to said surface, said middle cavity portion having a diameter greater than said distal and proximal cavity portions and forming an inwardly extending shoulder between said middle and proximal cavity portions, said anchor comprising:
an anchor body having a diameter substantially equal to said aperture and sized to pass therethrough;
a plurality of resilient, flexible legs attached to said anchor body, each of said legs having a free end resiliently biased to move substantially radially outwardly and engage said shoulder upon insertion of said anchor body into said middle cavity portion with said free ends facing said aperture, said legs being sufficiently flexible so as to be movable radially inwardly upon engagement of said legs with said cancellous layer during insertion of said anchor body into said distal cavity portion, said anchor thereby having substantially the same diameter as said distal cavity portion when sufficiently inserted into said distal cavity portion; and
an attachment extendable from said anchor body outwardly through said aperture for attaching said item to said anchor body.

23. An anchor according to claim 22, wherein said attachment comprises an elongated finger attached to said anchor body and extending lengthwise therefrom between said legs, said finger being sized so as to project above said surface for engagement with said item when said anchor body is positioned within said cavity.

24. An anchor according to claim 23, wherein said finger is adapted for engagement with a bone plate.

25. An anchor according to claim 22, wherein said attachment comprises a suture attached to said anchor body and extending therefrom, said suture for suturing soft tissue to said bone matter.

26. An anchor according to claim 22, wherein said anchor body comprises metal.

27. An anchor according to claim 26, wherein said metal is selected from the group consisting of titanium, nitinol, elgiloy and stainless steel.

28. An anchor according to claim 22, wherein said anchor body comprises a polymeric material.

29. An anchor according to claim 28, wherein said polymer is selected from the group consisting of polyester, polystyrene, polypropylene, PEEK, polyethylene and polytetrafluoroethylene.

30. An anchor according to claim 22, wherein said anchor body comprises biodegradable material.

31. An anchor according to claim 30, wherein said biodegradable material is selected from the group consisting of polyglycolic acid, polylactic acid, PLLA, PDO and PCL.

32. An anchor according to claim 22, wherein said anchor body comprises a head portion forming one end of said anchor body, and a tail portion extending lengthwise from said head portion and positioned between said legs, said legs extending from said head portion lengthwise along said tail portion.

33. An anchor according to claim 32, wherein said tail portion is adapted to extend outwardly through said aperture thereby comprising said attachment.

34. An anchor according to claim 32, wherein said head portion is tapered so as to facilitate insertion of said anchor body into said cavity.

35. An anchor according to claim 32, wherein said head portion has radially extending serrations preventing rotation of said anchor body within said cavity.

36. An anchor according to claim 32, wherein said tail portion has an eye therein for receiving a suture as said attachment.

37. An anchor according to claim 32, wherein said legs are adapted to be received within an end of a tube sized to hold said legs radially inwardly against said biasing, thereby allowing said anchor to pass through said aperture and into said proximal and distal cavity portions, said legs movable outwardly under said resilient biasing upon disengagement of said tube end from said legs.

38. An anchor according to claim 37, wherein each of said legs has an outwardly facing notch positioned at said free end thereof, said notches adapting said legs for being received within said tube.

39. An anchor according to claim 37 wherein each of said legs is tapered at said free end thereof, said tapering of said legs adapting said legs for being received within said tube.

40. An anchor according to claim 22, wherein said anchor body comprises a head portion forming one end of said anchor body, said legs extending lengthwise from said head portion to form another end of said anchor body, and an eye formed in said head for receiving a suture, said suture comprising said attachment.

41. An anchor according to claim 40, wherein said head portion is tapered so as to facilitate insertion of said anchor body into said cavity.

42. An anchor according to claim 40, wherein said legs are adapted to be received within an end of a tube sized to hold said legs radially inwardly against said biasing, thereby allowing said anchor body to pass through said aperture and into said proximal and distal cavity portions, said legs movable outwardly under said resilient biasing upon disengagement of said tube end from said legs.

43. An anchor according to claim 42, wherein each of said legs has an outwardly facing notch positioned at said free end thereof, said notches adapting said legs for being received within said tube.

44. An anchor according to claim 40, wherein each of said legs is tapered at said free end thereof, said tapering of said legs adapting said legs for being received within said tube.

45. An anchor according to claim 22, wherein each of said legs comprise a curved sheet of flexible material.

46. An anchor according to claim 45, wherein said legs are arcuately curved with concave portions facing inwardly of said anchor body.

47. An anchor according to claim 45, wherein said anchor body comprises a sheet of flexible material having an S-shaped cross section with oppositely disposed curved segments forming said legs.

48. An anchor kit for positioning an anchor within a cavity in a substrate to attach an item to said substrate, said cavity being accessible through an aperture in a surface of said substrate, said aperture having a smaller diameter than said cavity, an inwardly extending shoulder being positioned within said cavity, said anchor kit comprising:
an elongated insertion tool sized to pass through said aperture, said tool having mutually facing gripping surfaces positioned at one end;
an anchor body having a diameter sized to pass through said aperture;
an attachment fixed to said anchor body for attaching said item thereto, said attachment projecting away from said anchor body so that said attachment can extend outwardly through said aperture;
a plurality of resilient, flexible legs attached to said anchor body in spaced relation circumferentially therearound, each of said legs having a free end movable between a first position located within said anchor body diameter and a second position extending substantially radially outwardly beyond said anchor body diameter, said legs being resiliently biased into said second position; and
an engagement surface positioned on each of said legs at said free ends thereof facing radially outwardly therefrom, said engagement surfaces being engageable with said gripping surfaces of said insertion tool to retain said legs in said first position, said legs moving outwardly into said second position upon disengagement of said gripping surfaces from said engagement surfaces allowing said free ends to engage said shoulder and retain said anchor within said cavity.

49. An anchor kit according to claim 48, wherein said cavity comprises a distal portion positioned distally to said surface, a middle portion positioned between said distal portion and said surface, and a proximal portion positioned proximally to said surface, said middle cavity portion having a diameter greater than said distal and proximal cavity portions, said anchor kit further comprising:
a contact surface positioned on said anchor body between said legs and facing outwardly from said cavity; and
an elongated push piece sized to pass through said aperture and having an end engageable with said contact surface, said push piece for forcing said anchor body into said distal cavity portion, said substrate surrounding said distal cavity portion engaging and thereby moving said legs inwardly into said first position, said insertion tool being positionable within said cavity for engaging said gripping surfaces with said engagement surfaces and holding said legs in said first position thereby allowing removal of said anchor body from said cavity when said tube is removed therefrom.

50. An anchor kit according to claim 49, wherein said insertion tool comprises an elongated tube having a bore therethrough defined by an interior surface of said tube, portions of said interior surface at one end of said tube defining said gripping surfaces.

51. An anchor kit according to claim 49, wherein said tube has a diameter sized to pass through said aperture and substantially fill said proximal and distal cavity portions.

52. An anchor kit according to claim 51, wherein said push piece is sized to fit within said bore of said tube.

53. An anchor kit according to claim 49, wherein said attachment comprises a filamentary member attached to said anchor body and extending lengthwise therefrom.

54. An anchor kit according to claim 48, wherein each of said engagement surfaces are defined by a respective notch positioned in each of said free ends of said legs.

55. An anchor kit according to claim 48, wherein each of said legs are tapered at said free ends thereby defining said engagement surfaces.

56. An anchor kit for removably positioning an anchor within a cavity in a substrate, said cavity being accessible through an aperture in a surface of said substrate and having a distal portion positioned distally to said surface, a middle portion positioned between said distal portion and said surface, and a proximal portion positioned proximally to said surface, said middle cavity portion having a diameter greater than said distal and proximal cavity portions, an inwardly extending shoulder being positioned between said middle and proximal cavity portions, said anchor kit comprising:
an anchor body having a plurality of lengthwise extending legs attached thereto, each of said legs having a free end resiliently biased to move outwardly and engage said shoulder upon insertion of said anchor body into said middle cavity portion, said legs being inwardly movable upon insertion of said anchor body into said distal cavity portion;
a flexible filamentary member having one end attached to said anchor body and an opposite end extending away therefrom;
a contact surface positioned between said legs of said anchor body and facing outwardly from said cavity;
an elongated tube sized to pass through said aperture and substantially fill said proximal and distal cavity portions, said tube having a longitudinal bore therethrough sized to receive said free ends of said legs when said anchor body is positioned within distal cavity portion; and
an elongated rod sized to pass through said aperture and having an end engageable with said contact surface, said rod for forcing said anchor body into said distal cavity portion, said substrate surrounding said distal cavity portion engaging and thereby moving said legs inwardly, said tube being positionable within said cavity with said bore aligned with said anchor body, said free ends of said legs being movable into said bore, said legs being constrained inwardly by said tube, said tube being removable from said cavity with said anchor body therein.

57. An anchor kit according to 56, wherein said substrate comprises a relatively hard layer of material overlying a relatively soft layer of material, said relatively hard layer forming said surface and having said aperture therein, said distal cavity portion being positioned within said relatively soft layer, said proximal cavity being positioned within said relatively hard layer, said legs being sufficiently flexible so as to be moved inwardly and engageable with said tube upon engagement with said relatively soft layer comprising said substrate.

58. An anchor kit according to claim 57, wherein said substrate comprises bone matter having a cortical layer overlying a cancellous layer, said cortical layer comprising said relatively hard layer, said cancellous layer comprising said relatively soft layer.

59. An anchor kit according to claim 58, wherein said filamentary member comprises a suture attached to said anchor body and extending lengthwise therefrom, said suture for suturing soft tissue to said bone matter.

60. A method of inserting an anchor in a cavity in a substrate, said cavity being accessible through an aperture in a surface of said substrate and having a distal portion positioned distally to said surface, a middle portion positioned between said distal portion and said surface, and a proximal portion positioned proximally to said surface, said middle portion having a diameter greater than said distal and proximal portions, an inwardly extending shoulder being positioned between said middle and proximal cavity portions, said anchor comprising an elongated anchor body having a plurality of lengthwise extending legs, each of said legs having a free end resiliently biased to move outwardly and engage said shoulder upon insertion of said anchor body into said middle cavity portion, said legs being held inwardly upon engagement of said free ends with an end of an insertion tool so as to be passable through said aperture, said method comprising the steps of:
engaging said free ends of said legs with said end of said insertion tool thereby holding said legs inwardly;
inserting said anchor and said insertion tool through said aperture and into said cavity;
disengaging said insertion tool end from said legs, said legs moving outwardly under said resilient biasing; and
withdrawing said insertion tool from said cavity.

61. A method of removing an anchor positioned in a cavity in a substrate, said cavity being accessible through an aperture in a surface of said substrate and having a distal portion positioned distally to said surface, a middle portion positioned between said distal portion and said surface, and a proximal portion positioned proximally to said surface, said middle portion having a diameter greater than said distal and proximal portions, an inwardly extending shoulder being positioned between said middle and proximal cavity portions, said anchor comprising an elongated anchor body having a plurality of lengthwise extending legs, each of said legs having a free end resiliently biased to move outwardly and engage said shoulder upon insertion of said anchor body into said middle cavity portion, said legs being inwardly movable upon insertion of said anchor body into said distal cavity portion, said method comprising the steps of:
pushing said anchor body into said distal cavity portion sufficiently to move said legs inwardly;
inserting an elongated tool into said cavity, said tool having an end for receiving said legs;
engaging said tool end with said legs, said tool receiving and constraining said legs from moving outwardly; and
withdrawing said tool from said cavity with said legs of said anchor body engaged with said tool.
